(19) United States
(12) Patent Application Publication
(10) Pub. No.: US 2024/0123008 A1
(43) Pub. Date: Apr. 18, 2024

(54) USE OF A FLAVANOID EXTRACT OBTAINED FROM THE SPECIES TALIPARITI ELATUM SW, FORMULATIONS COMPRISING SAME AND TREATMENT METHOD

(71) Applicant: CENTRO DE INVESTIGACION Y DESARROLLO DE MEDICAMENTOS (CIDEM), Habana (CU)

(72) Inventors: Maylin WONG GUERRA, La Habana (CU); Jeney RAMIREZ SANCHEZ, La Habana (CU); Luis Arturo FONSECA FONSECA, La Habana (CU); Yanier NÚÑEZ FIGUEREDO, La Habana (CU); Alejandro Saul PADRON YAQUIS, La Habana (CU); Loraine Licet INFANTE BLANCO, La Habana (CU); Jacqueline Aylema ROMERO DIAZ, Winchester Hampshire (GB); Maydel BARRIOS SARMIENTO, La Habana (CU); Yanay MONTANO PEGUERO, Artemisa (CU)

(73) Assignee: CENTRO DE INVESTIGACION Y DESARROLLO DE MEDICAMENTOS (CIDEM), Habana (CU)

(21) Appl. No.: 17/768,532

(22) PCT Filed: Nov. 13, 2020
(86) PCT No.: PCT/CU2020/050005

§ 371 Date: Apr. 13, 2022

(30) Foreign Application Priority Data
Nov. 14, 2019 (CU) 2019-0091

(51) Int. Cl. A61K 36/185; A61K 31/7048; A61K 47/32; A61K 47/38; A61P 25/00

(52) U.S. Cl. CPC A61K 36/185; A61K 31/7048; A61K 47/32; A61K 47/38; A61P 25/00; A61K 2236/33; A61K 2236/35; A61K 2236/39

(57) ABSTRACT

This invention is related to the field of healthcare and especially to the treatment of memory disorders. The invention consists in the preparation of oral pharmaceutical formulations from an extract of flavonoids obtained from the species Talipairti elatum Sw., which acts improving the different types of memory, both in the short and long-term associated with different pathologies and with population aging in humans.

### FIELD OF INVENTION

The present invention is related to the health field and especially to the use of an extract rich in flavonoids obtained from Talipariti elatum Sw and of pharmaceutical formulations containing it, in the treatment of diseases with memory disorders.

### Background of the Invention

Hibiscus elatus or Talipariti elatum Sw. or majagua is an endemic plant from Cuba, Jamaica, United States of America, Virgin Islands, Puerto Rico and Martinique. In humid areas, it grows at a wide range of elevations, and is very often used in reforestation. It is the national tree of Jamaica.

In Cuba, there are two species of plants known as “majagua”, Hibiscus tiliaceus, a small and branching tree and the common majagua or blue majagua, a tree that reaches up to about 20 m in height that corresponds to the Talipariti elatum Sw., a species considered endemic; its flowers in Cuba are highly appreciated by traditional medicine as expectorants and anti-asthmatic. Phytochemical studies carried out in our country, confirm the presence, in the petals of the flower of this specie, of the o-glycoside flavonoid Gossypitrin and suggest the presence of other flavonoids.

Gossypitrin, a major component present in the flowers of this species, is a flavan-3-ol glycosylate in carbon 7 of the A ring and it also has two free OH groups in position 5 and position 8 in the A ring, a catechol group in positions 3′ and 4′ in the B ring, and a free OH group in carbon 3 of the C ring. Its chemical structure is shown below

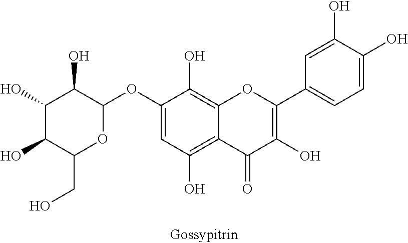 [SMILES: O=c1c(O)c(-c2ccc(O)c(O)c2)oc2c(O)c(OC3OC(CO)[C@@H](O)[C@H](O)[C@H]3O)cc(O)c12]

The Author's Certificate No. CU22484 A1 relates to the field of healthcare and especially to allergology. This invention describes the preparation of a syrup that acts in the stabilization of the functions of the bronchial tree, both in a preventive and abortive way of an asthmatic attack. Such syrup is obtained from plant extracts: Musa paradisiaca, Hibiscus elatus and Aloe barbadensi, which provide different active principles, and it is applicable in low respiratory obstructive syndrome.

In other documents non-classified as patents, some medicinal properties of Hibiscus elatus (Majagua) are reported, among them: to relieve gastrointestinal and respiratory ailments; to soothe rheumatic and muscular pains. Its leaves cure hemorrhoids; it also has analgesic properties for the ears and bronchi, as well as emollients, as it softens tumors. An expectorant and anti-asthmatic syrup is made with the flower and its flowers are widely used to prevent hair loss, and improve its shine; red flowers are used to blacken hair, and yellow ones for light hair. Some beneficial effects are also reported that improve the appearance of the skin and promote the healing of bones and cartilage lesions, and the formation of bones and cartilage. However, there are no reports suggesting or relating pharmaceutical formulations of phenolic compounds isolated from Talipariti elatum Sw. for the treatment of any type of dementia.

The inventors of this application have surprisingly found that a crude flavonoid obtained from Talipariti elatum Sw. and its pharmaceutical compositions may be useful in the treatment of diseases in which there is impairment of short-term and long-term memory.

### BRIEF DESCRIPTION OF THE INVENTION

A first object of this invention is the use of an extract rich in flavonoids obtained from Talipariti elatum Sw., in the treatment of short and long-term memory disorders.

A second object of this invention refers to providing pharmaceutical compositions including an extract according to the first object of invention, and one or more pharmaceutically acceptable excipients.

A particular aspect of these pharmaceutical compositions is that they may be used in the treatment of short- and long-term memory disorders.

Such compositions may be indicated for oral administration in either solid or liquid form.

A third object of the present invention is to provide a method for the treatment of memory disorders to a subject who needs it, where such method involves the administration to said subject of a therapeutically effective dose of an extract according to the first object of invention, or a pharmaceutical composition according to the second object of invention.

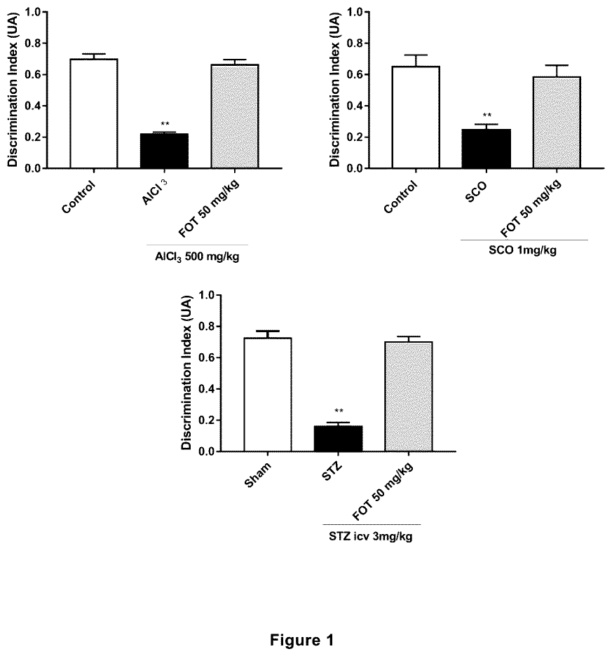

FIG. 1. Effect of flavonoid extract from petals of Taliparitis elatum Sw. (ET) on recognition memory in different cognitive disorder schemes.

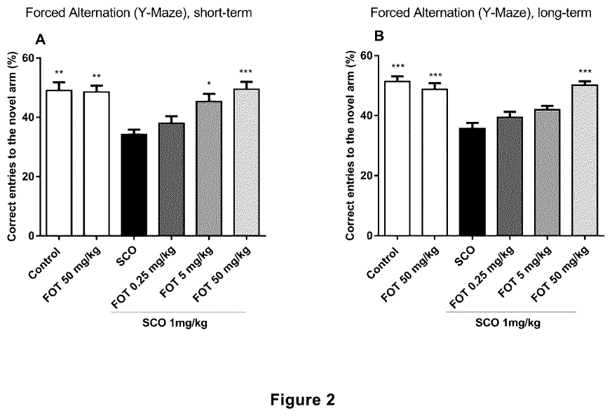

FIG. 2. Effect of oral formulations of flavonoid extract from petals of Talipariti elatum Sw. (FOT) on short and long-term spatial memory, against cognitive impairment induced by scopolamine (SCO) (1 mg/kg) in rats.

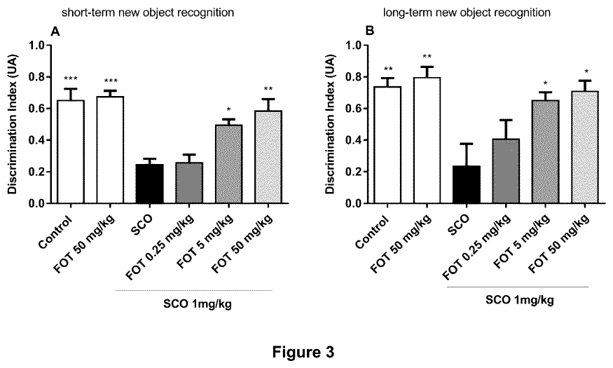

FIG. 3. Effect of oral formulations of flavonoid extract from petals of Talipariti elatum Sw. (FOT) on short and long-term recognition memory against cognitive impairment induced by scopolamine (SCO) (1 mg/kg) in rats.

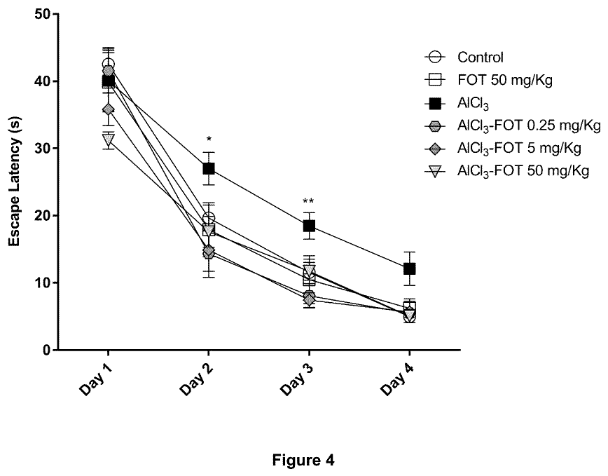

FIG. 4. Effect of oral formulations of flavonoid extract from petals of Talipariti elatum Sw. (FOT) on spatial memory against cognitive disorder induced by chronic administration of aluminum chloride (AlCl3) in rats.

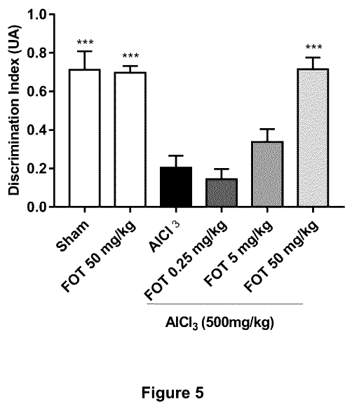

FIG. 5. Effect of oral formulations of flavonoid extract from petals of Talipariti elatum Sw. (FOT) on the novel object recognition memory in cognitive disorder model induced by aluminum chloride (AlCl3) in rats.

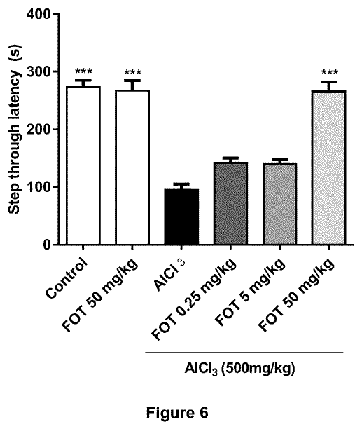

FIG. 6. Effect of oral formulations of flavonoid extract from petals of Talipariti elatum Sw. (FOT) on associative memory in the cognitive disorder model induced by aluminum chloride (AlCl3) in rats.

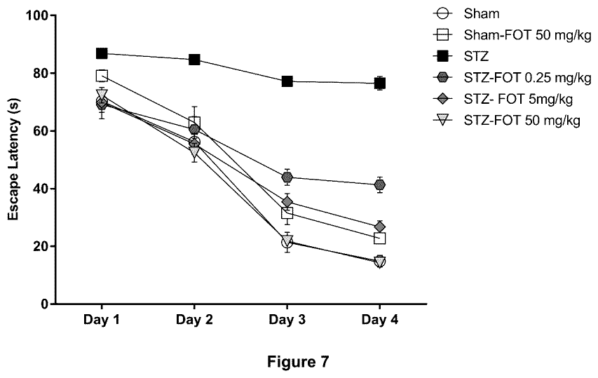

FIG. 7. Effect of oral formulations of flavonoid extract from petals of Talipariti elatum Sw. (FOT) on spatial memory compared to cognitive disorder induced by icv-STZ administration in rats.

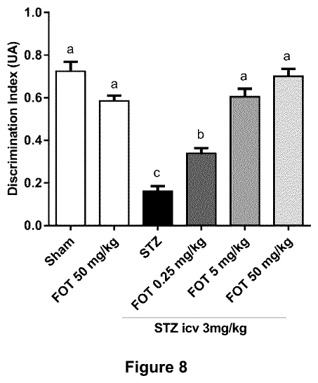

FIG. 8. Effect of oral formulations of flavonoid extract from petals of Talipariti elatum Sw. (FOT) on novel object recognition memory.

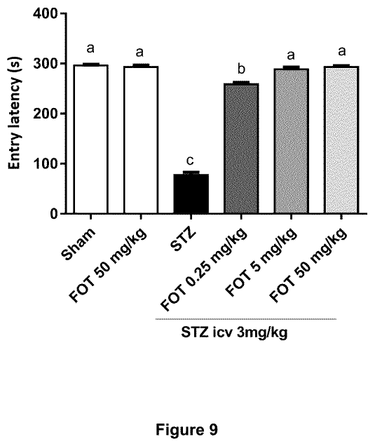

FIG. 9. Effect of oral flavonoid extract formulations from the petals of Talipariti elatum Sw. (FOT) on associative memory.

### DETAILED DESCRIPTION OF THE INVENTION

In the present invention the inventors surprisingly found that the use of an extract obtained from petals of the species Talipariti elatum Sw. has biological activity in the treatment of memory disorders.

Consequently, a biologically active extract and its pharmaceutical compositions are provided.

When evaluating the extract in different dementia models (damage induced by scopolamine, aluminum chloride and streptozotocin), a dose-dependent protective effect was observed in all cases. The results corresponded to those obtained for the oral formulations described in the example of realization number 5.

The extract of the invention may be administered in accordance with this invention in the form of pharmaceutical compositions, which include one or more pharmaceutically acceptable carriers, excipients or diluents.

The specific dose of a composition to be used on a particular individual will depend on a variety of factors including, for example, age, body weight, health condition and diet of the individual to be treated, time of administration, stability of the extract and its combination with any other treatment.

A granular formulation is proposed that contains:

A therapeutically effective amount of flavonoid extract from the petals of Talipariti elatum Sw. (according to examples of realization 1 and 2); from 2 up to 6% in weight, with respect to the active ingredient, of a hydrophilic binding agent; from 16 up to 80% in weight, with respect to the weight of the active ingredient, of a diluting agent; from 1 up to 9% in weight, with respect to the weight of the active ingredient of a disintegrating agent.

The preferred binders are starch in a proportion of at least 5.6% in weight to the flavonoid extract from the petals of Talipariti elatum Sw, and
polyvinylpyrrolidone, in a proportion of approximately 2.3% in weight to the flavonoid extract from the petals of Talipariti elatum Sw.

The starch may be from rice, wheat or corn. The preferred starch is cornstarch and may be a partially pre-gelatinized starch, such as 1500 starch (Colorcon). In addition to its function as a binder, a part of starch, especially in the capsules of 25 and 50 mg flavonoid extract from the petals of Talipariti elatum Sw. may perform diluent functions.

Any suitable polyvinylpyrrolidone capable of acting as a binder may be used. Polyvinylpyrrolidone may be a linear 1-vinyl-2-pyrrolidone polymer that has an average molecular weight of approximately 40000, such as Povidone K30 (ISP Pharmaceutical).

The preferred diluent agent is microcrystalline cellulose or mixtures of microcrystalline cellulose and starch.

The preferred disintegrating agent is sodium starch glycolate.

The granular formulations of this invention are obtained from a process involving wet granulation:

The flavonoid extract from the petals of Talipariti elatum Sw. the diluting agent, and optionally the disintegrating agent, in the presence of a hydrophilic binding agent, and optionally mix with the same or another disintegrating agent and/or also with other pharmaceutically acceptable auxiliary agents.

Pharmaceutically acceptable auxiliary agents, preferred in the present invention are, among others:
Lubricants, such as sodium benzoate, used in a range of 0.25-5% with respect to the weight of flavonoid extract from the petals of Talipariti elatum Sw.

Liquid oral formulations contain, in addition to the flavonoid extract from the petals of Talipariti elatum Sw, at least one liquid vehicle to dissolve or suspend the flavonoid extract from the petals of Talipariti elatum Sw, moisturizing agents, Sweetening agents, flavoring substances, preservative agents and acidulants to neutralize the flavor of the flavonoid extract from the petals of Talipariti elatum Sw.

Liquid oral formulations which are likely to be used repeatedly should contain a soluble preservative agent. The concentration of preservatives may vary from 0.05% to 1% w/v, particularly from 0.1% to 0.5% w/v and in a very particular way it is approximately 0.2% w/v. The preferred preservative is sodium benzoate.

To improve the aroma of the flavonoid extract from the petals of Talipariti elatum Sw., one or more intense Sweetening agents such as saccharin, saccharin sodium, potassium or calcic or sodium cyclamate may be used as an option. The concentration of the Sweetening agent may range from 0.05% to 0.5% w/v and in particular it is approximately 0.2% w/v. Optionally, the palatability of these solutions may also be optimized by the addition of one or more flavoring substances. Suitable flavoring substances are fruit flavors. The strawberry aroma has been found to produce very satisfactory aroma masking results. The total concentration of flavoring substances may vary from 0.01% to 0.2% w/v, preferably from 0.02% to 0.1% w/v and in particular it is approximately 0.025% w/v. The total concentration of flavoring substances may vary from 0.01% to 0.2% w/v, preferably from 0.02% to 0.1% w/v and in particular, it is approximately 0.025% w/v.

The preferred acidulant agent is citric acid.

An oral liquid composition in accordance with the present invention comprises:
0.5% to 1.5% p/v of flavonoid extract from the petals of Talipariti elatum Sw.
0.1% to 0.5% w/v of preservatives
0.01% to 0.2% w/v of flavourings
0.05% to 0.5% w/v of Sweeteners
0.15% to 0.5% w/v of acidulants 5% to 15% w/v of humectants
30% to 80% w/v of hydrogenated glucose syrup and purified water, in sufficient quantity up to 100%, as liquid vehicles

The most preferred liquid oral composition in accordance with this invention includes:

1% w/v (0.01 g/mi) flavonoid extract from the petals of Talipariti elatum Sw., as active ingredient 0.2% w/v (0.002 g/mi) sodium benzoate, as preservative 0.025% w/v (0.00025 g/mi) strawberry flavouring, as flavouring 0.2% w/v (0.002 g/mi) sodium saccharin, as Sweetener 0.35% w/v (0.002 g/mi), 0035 g/mi) of citric acid, as an acidifier 10% w/v (0.1 g/ml) of glycerol, as a humectant and 64% (0.64 g/mi) of hydrogenated glucose syrup and purified water, in sufficient quantity up to 100% (1 mL), as liquid vehicles.

The liquid formulations of this invention are obtained from a process that comprises:

Dissolve the glycerol and strawberry flavouring in one part of water, and then add the flavonoid extract from the petals of Talipariti elatum Sw, hydrogenated glucose syrup, citric acid, sodium benzoate and sodium saccharin, stir for a time and bring up to the given volume.

In FIG. 2 the results are expressed as a percentage of correct entries to the new arm of the Y maze, in the Y maze forced alternation test. A) Correct entries (%) in a short-term memory paradigm. B) Correct entries (%) in a long-term memory paradigm. Data are expressed as mean±DE (n=7, per group). For statistical analysis, one-way ANOVA and Tukey test p<0.05 was performed.

While in FIG. 3, the results are expressed as index of discrimination between familiar and novel objects, evaluated in the Novel Object Recognition test. A) Discrimination index (AU) in a short-term memory paradigm. B) Discrimination index (AU) in a long-term memory paradigm. Data are expressed as mean±DE (n=7, per group). Two-way ANOVA and Bonferroni p<0.05 tests were performed for statistical analysis.

AU: Arbitrary Units.

FIGS. 2 and 3 show the experimental results, where both the forced alternation Y maze test that evaluates spatial memory, and the novel object recognition memory test, 5 and 50 mg/kg doses of the formulation protected from damage caused by scopolamine to short and long-term memory.

In FIG. 4, the results are expressed as escape latency during training days (learning curve). Data are expressed as mean±DE (n=7, per group). Two-way ANOVA and Bonferroni p<0.05 tests were performed for statistical analysis.

In FIG. 5, the results are expressed as a discrimination index of the total time exploring N object with respect to F. The data are expressed as mean±DE (n=7, per group). For statistical analysis, one-way ANOVA was performed followed by Tukey's multiple comparison test p<0.05.

While in FIG. 6 the results are expressed as entry latency (time it takes the animal to move to the dark zone of a maximum of 300 seconds). The data are expressed as mean±DE (n=7, per group). For statistical analysis, one-way ANOVA was performed followed by Tukey's multiple comparison test p<0.01.

FIG. 4 shows the effect of the formulation on the spatial reference memory, where it was found that the 50 mg/kg dose protects against the progression of cognitive disorder induced by aluminum chloride, while 0.25 and 5 mg/kg doses did not prevent the progression of disorder. Similarly, it was observed that only the 50 mg/kg dose of the formulation protected from the deterioration progress of novel recognition memory (FIG. 5) and associative emotional memory (FIG. 6).

In FIG. 7, the results are expressed as escape latency during training days (learning curve). The data are expressed as mean±DE (n=7, per group). A two-way ANOVA and Bonferroni test p<0.01 were performed for statistical analysis.

In the FIG. 8 the results are expressed as a discrimination index of the total time exploring object N with respect to F. The data are expressed as mean±DE (n=7, per group). For statistical analysis, a one-way ANOVA was performed followed by Tukey's multiple comparison test p<0.05.

While in FIG. 9, the results are expressed as input latency (the time it takes the animal to move to the dark zone of a maximum of 300 seconds). The data are expressed as mean±DE (n=7, per group). For statistical analysis, one-way ANOVA was performed followed by Tukey's multiple comparison test. Different letters differ from each other for p<0.01.

FIG. 7 shows the experimental results of the Morris water maze. The 5 and 50 mg/kg doses of the formulation protected the spatial memory from damage caused by icy STZ, preventing the establishment and progression of alterations that lead to the appearance of cognitive disorders. FIG. 8 shows the results of the novel object recognition test, where 5 and 50 mg/kg doses also protect the novel recognition memory against damage caused by icy STZ. On the other hand, the study of the emotional-associative memory similarly showed that 5 and 50 mg/kg doses protect from the damage caused by icy STZ, verifying a consistent memory of the negative reinforcement in the animals treated with the formulation in comparison with the STZ group (FIG. 9). From these results, we can observe that the oral formulations of flavonoid extract from the petals of Talipariti elatum Sw. protect from damage to different types of memory caused by icy STZ, on a dose-dependent basis.

In addition, methods of treatment for memory disorders are provided. Such methods are applied in the treatment of different diseases, including different types of dementias.

### EXAMPLES

### Example 1. Flavonoid Extract Obtained from the Petals of Talipariti elatum Sw

To obtain the flavonoid extract, 40 g of previously dried Talipariti elatum Sw. petals were weighed and transferred to a flat-bottomed distillation flask containing 700 ml of n-hexane. The reflux was made at 60° C. in a water bath for 2 hours, the solvent was discarded and the material residue was left to dry at room temperature for 24 hours. Subsequently, acetone was added to the dry residue 80% enough to maintain a vegetable material ratio:solvent 1:18.97. Again, a reflux was applied in water bath at 60° C. during 2 hours, it was hot filtered and the same process was carried out 2 times more with the remaining residue in the bottom of the flask.

The extracts obtained were transferred to a 1 L round-bottom distillation flask and the starting volume was taken to ⅕, subjecting the sample to 60° C. under reduced pressure. It was transferred to a separating funnel and 5 liquid extractions were carried out: liquid (1:1) with n-butanol. The organic phase was collected in a previously tared distillation flask and dried under reduced pressure. The flask was weighed again after evaporation of the solvent to express the amount of total flavonoid extract obtained.

### Example 2. Optimization of the Flavonoid Extraction Process from the Petals of Talipariti elatum Sw

Soxhlet extractions were carried out until exhaustion of the plant material with several solvents previously described in the literature for the extraction of flavonoids and anthocyanins: ethanol, methanol and acetone at various concentrations. The solvent with which a higher concentration of the majority of metabolites was obtained was chosen, quantified by HPLC and total flavonoid methods. The temperature was set at 60° C. to ensure the stability of the metabolites.

The optimization of the extraction parameters was carried out by means of a Response Surface methodology. A Box-Becken design of 15 experiments with 3 factors was executed at two tiers. The Stargraphic software version 5.1 was used to generate the experimental design, statistical analyses and the regression model. The response variable determined was total flavonoids.

The highest content of Gossypitrin and total flavonoids for the different solvents tested was found with 80% Acetone.

The quantification of the flavonoids, as well as the values for each one of the experiments for optimizing the rest of the variables studied are described in Table 1.

TABLE 1
Experimental design and flavonoid values obtained for each one.

| | Amount of plant | Number of | Extraction time | Total |
|---|---|---|---|---|
| Expt. | material (%) | extractions | (min) | flavonoids (mg/g) |
| 1 | 5 | 2 | 2 | 37.81 |
| 2 | 5 | 3 | 1 | 39.72 |
| 3 | 3 | 3 | 0.5 | 45.02 |
| 4 | 2.5 | 2 | 0.5 | 39.13 |
| 5 | 3 | 3 | 2 | 46.79 |
| 6 | 5 | 2 | 0.5 | 37.09 |
| 7 | 3 | 1 | 2 | 32.37 |
| 8 | 2.5 | 3 | 1 | 47.88 |
| 9 | 2.5 | 2 | 2 | 44.78 |
| 10 | 3 | 2 | 1 | 41.19 |
| 11 | 5 | 1 | 1 | 24.81 |
| 12 | 2.5 | 1 | 1 | 32.37 |
| 13 | 3 | 2 | 1 | 39.96 |
| 14 | 3 | 1 | 0.5 | 28.53 |
| 15 | 3 | 2 | 1 | 40.83 |

The equation describing the process (Flavonoids (mg/g)=40.66+3.09125X3+7.66625X1+1,4975X2−1.47X32+0.15X3X1+1.2325X3X2−2.995X32-0.5175X1X2+0.5125X22) and the factors that showed a significant influence on the amount of flavonoids obtained, which are shown in FIG. 1, were obtained. The value of R 2=0.989463 indicates that the model thus adjusted accounts for 98.9463% of the variability in the amount of flavonoids. The adjusted R-square statistic, which is more suitable for the comparison of different numbers of independent variables, is 97.0497%.

The 3 variables analyzed showed significant influence on the flavonoid content obtained, as well as the square of the number of extractions.

The extraction was repeated in triplicate at the optimum point, obtaining a total flavonoid content of 4.95 g/100 g of petals.

### Example 3. Effect of Flavonoid Extract from Talipariti elatum Sw. Petals on Different Memory Disorders

The obtained extracts were evaluated in different models of dementia for the purpose of demonstrating their neuroprotective properties for the later development of oral formulations. Scopolamine-induced, aluminum chloride and streptozotocin-induced disorder models were used. In all cases, experimental methodologies were used to evaluate short and long-term memory and recognition, associative and working memories.

### Example 4. Pharmaceutical Compositions from Flavonoid Extract from Talipariti elatum Sw. Petals for Use in Humans

The following embodiments are given for the purpose of providing the person with expertise in the subject matter with a sufficiently clear and complete explanation of this invention, but they should not be regarded as limitations on the essential aspects of the subject matter of the invention, as set out in the previous sections of this description.

1: Tablet Formulations

| Product | Mg/tablet |
|---|---|
| Extract from Talipariti elatum Sw. | 50 |
| Microcrystalline Cellulose | 40 |
| Povidone | 5 |
| Sodium starch glycolate | 5 |
| Magnesium stearate | 3 |
| Total | 103 |

Flavonoid extract is mixed from the petals of Talipariti elatum Sw., microcrystalline cellulose and sodium starch glycolate and they are screened together through a 0.8 mm mesh sieve. A solution of polyvinylpyrrolidone is added to water or a mixture of water and ethanol and mixed over a period of time. The wet mixture is passed through a granulator and the granulate obtained is dried at an appropriate temperature for the time required. The dry granulate is calibrated through a sieve and mixed thoroughly with titanium dioxide and magnesium stearate. Free-flowing granule powder is obtained.

The granulate is compressed in a compression machine. The obtained tablets may be coated.

2: Capsule Formulations

Flavonoid extract is mixed from the petals of Talipariti elatum Sw., microcrystalline cellulose and cornstarch, ethanol is added and mixed for some time. The wet mixture is passed through a granulator and the granulate obtained is dried for the time required. The dry granulate is calibrated through a sieve and mixed thoroughly with sodium glycolate starch and magnesium stearate. Free-flowing granule powder is obtained.

The granulate is used to fill the capsules.

As an alternative process, flavonoid extract is mixed from the petals of Talipariti elatum Sw. and microcrystalline cellulose, a starch paste is prepared, added to the above mixture and mixed over a period of time. The wet mixture is passed through a granulator and the granulate obtained is dried for the time required. The dry granulate is calibrated through a sieve and mixed thoroughly with starch sodium glycolate and magnesium stearate. Free-flowing granule powder is obtained. The granulate is used to fill the capsules.

| Product | Mg/capsule |
|---|---|
| Extract from Talipariti elatum Sw. | 100 |
| Microcrystalline Cellulose | 25 |
| Corn Starch | 55 |
| Sodium starch glycolate | 7 |
| Magnesium stearate | 1 |
| Total | 188 |

3: Liquid Formulations

Stir a mixture of glycerol, strawberry aroma and in part of the water. The flavonoid extract is then added from the petals of Talipariti elatum Sw., hydrogenated glucose syrup, citric acid, sodium benzoate and sodium saccharin, it is stirred over a period of time, and made up to a certain volume and dosed in vials.

| Product | Formula per 200 mL | |
|---|---|---|
| Extract from Talipariti elatum Sw. | 1 | g |
| Hydrogenated glucose syrup | 64 | g |
| Glycerol | 10 | g |
| Citric acid | 0.3 | g |
| Sodium benzoate | 0.2 | g |
| Sodium saccharin | 0.2 | g |
| Aroma of orange | 0.02 | g |

| | |
|---|---|
| Purified water | c.s.p 200 mL |

The liquid formulations of this invention are obtained from a process comprising: dissolving the glycerol and strawberry flavouring in part of the water, and then adding the flavonoid extract from the petals of Talipariti elatum Sw., hydrogenated glucose syrup, citric acid, sodium benzoate and sodium saccharin, stirring for a time and making up to the given volume.

### Example 5. Effect of Oral Formulations of Flavonoid Extract from Petals of Talipariti elatum Sw. (FOT) According to Exemplary Embodiment 4 in Different Memory Disorders

1. Effect of Oral Formulations of Flavonoid Extract from the Petals of Talipariti elatum Sw. (FOT) on Short-Term and Long-Term Scopolamine-Induced Memory Disorder.

In order to evaluate the protective effect on short and long-term memory of oral flavonoid extract formulations from the petals of Talipariti elatum Sw., the scopolamine-induced dementia model was used in adult male Wistar rats. The formulation was administered in doses of 0.25, 5 and 50 mg/kg body weight for 7 days before inducing cognitive disorder with scopolamine 1 mg/kg body weight. A short-term paradigm was used in which scopolamine was administered and 40 minutes later the training phase of the behavioral test was performed (Y maze of forced alternation or recognition of novel objects), after 2 hours the evaluation phase of the behavioral test was performed. Another paradigm was used to evaluate the protective effect on long-term memory, with the difference that between the training and evaluation phases there were 24 hours.

2. Effect of Oral Formulations of Flavonoid Extract from the Petals of Talipariti elatum Sw. (FOT) on the Disorder of Different Types of Memory Induced by Aluminum Chloride.

The protective effect of oral flavonoid extract formulations from Talipariti elatum Sw. petals on different memory types affected by chronic administration (30 days) of aluminum chloride (500 mg/kg) in adult male Wistar rats was evaluated. The formulation was administered in doses of 0.25, 5 and 50 mg/kg body weight for 15 days; 15 days after the beginning of the administration of aluminum chloride (orally). Three different behavioral tests were used to evaluate different types of memory: Morris water maze to study spatial reference memory, object recognition to evaluate novel memory recognition, and passive avoidance test to evaluate emotional-associative memory.

3. Effect of Oral Formulations of Flavonoid Extract from the Petals of Talipariti elatum Sw. (FOT) on the Disorder of Different Types of Memory Induced by Streptozotocin.

To evaluate the protective effect on short and long-term memory of oral formulations of flavonoid extract from the petals of Talipariti elatum Sw. the model of dementia induced by intracerebroventricular administration of streptozotocin (icy STZ) in adult male C57BL6 mice was used. The formulation was administered in doses of 0.25, 5 and 50 mg/kg body weight for 13 days, 1 day after induction of disorder with icy STZ (3 mg/kg body weight). Three different behavioral tests were used to evaluate different types of memory: object recognition to evaluate novelty recognition memory, Morris water maze to study spatial reference memory, and passive avoidance test to evaluate emotional-associative memory.

1. A flavonoid extract from the petals of Talipariti elatum Sw. for use in the treatment of memory disorders.

2. A pharmaceutical formulation for oral administration comprising a therapeutically effective amount of the extract according to claim 1, together with:
at least one hydrophilic binding agent,
at least one diluent agent and
at least one disintegrating agent.

3. The pharmaceutical formulation according to claim 2 characterized by being in solid form.

4. The pharmaceutical formulation according to claim 3 characterized as being in the form of granulates.

5. The pharmaceutical formulation according to claim 3 characterized as being in tablet form.

6. The pharmaceutical formulation according to claim 3 characterized as being in capsule form.

7. The pharmaceutical formulation according to claim 2 characterized because the hydrophilic binding agent is present between 2 and 6% in weight with respect to the weight of the flavonoid extract from the petals of Talipariti elatum Sw., the diluting agent is present in 16 and 80% in weight with respect to the weight of the flavonoid extract from the petals of Talipariti elatum Sw., and the disintegrating agent is present between 1% and 9% in weight with respect to the weight of the flavonoid extract from the petals of Talipariti elatum Sw.

8. The pharmaceutical formulation, according to claim 2, characterized by the hydrophilic binding agent being starch or polyvinylpyrrolidone, the diluting agent being microcrystalline cellulose or mixtures thereof with starch, and the disintegrating agent being sodium starch glycolate.

9. The pharmaceutical formulation, in accordance with claim 8, comprises 52 to 72% in weight of flavonoid extract from the petals of Talipariti elatum Sw., 4 to 29% in weight of starch, 13 to 18% in weight of microcrystalline cellulose, 4 to 6% in weight of sodium starch glycolate.

10. The pharmaceutical formulation, in accordance with claim 8, comprises 52 to 85% in weight of flavonoid extract from the petals of Talipariti elatum Sw., 1 to 2% in weight of polyvinylpyrrolidone, 10 to 46% in weight of microcrystalline cellulose, 1 to 6% in weight of starch sodium glycolate.

11. The pharmaceutical formulation according to claim 8 is characterised because the hydrophilic binding agent is starch and is present in at least 5% in weight of the flavonoid extract from the petals of Talipariti elatum Sw, the diluting agent is microcrystalline cellulose and is present between 20 and 30% in weight of the flavonoid extract from the petals of Talipariti elatum Sw. and the disintegrating agent is sodium starch glycolate and is present in approximately 8% in weight of the active ingredient.

12. The pharmaceutical formulation according to claim 8 is characterized because the hydrophilic binder agent is polyvinylpyrrolidone and is present between 2 and 3% in weight with respect to the weight of the flavonoid extract from the petals of Talipariti elatum Sw., the diluting agent is microcrystalline cellulose and is present between 12 and 25% in weight with respect to the weight of the flavonoid extract from the petals of Talipariti elatum Sw. and the disintegrating agent is sodium starch glycolate and is present between 1 and 3% in weight with respect to the weight of the active ingredient.

13. The pharmaceutical formulation according to claim 2, characterized because it presents in its composition, additionally, a lubricating compound, preferably magnesium stearate.

14. The pharmaceutical formulation according to claim 2 characterized as being in liquid form.

15. The pharmaceutical formulation for oral administration, according to claim 14, comprising a therapeutically effective amount of flavonoid extract from the petals of pharmaceutically acceptable Talipariti elatum Sw. characterized by the fact that it also contains: at least one preservative, at least one aromatizer, at least one Sweetener, at least one acidulant, at least one moisturizer and at least one liquid vehicle.

16. The pharmaceutical formulation in accordance with claim 15 containing: 0.5% to 1.5% w/v flavonoid extract from the petals of Talipariti elatum Sw. 0.1% to 0.5% w/v of preservatives 0.01% to 0.2% w/v of aromatizers 0.05% to 0.5% w/v of Sweeteners 0.15% to 0.5% w/v of acidulants 5% to 15% w/v of moisturizers 30% to 80% w/v of hydrogenated glucose syrup purified water, a sufficient amount up to 100%.

17. The pharmaceutical formulation according to claim 15, characterized because the aromatizing agent is the strawberry aroma.

18. The pharmaceutical formulation according to any of claim 15, in which sodium benzoate is preferably used as a preservative agent.

19. The pharmaceutical formulation according to claim 15 where citric acid is used as an acidulant.

20. The pharmaceutical formulation according to claim 17 containing 1% (0.01 g/ml) flavonoid extract from the petals of Talipariti elatum Sw., 0.2% (0.002 g/ml) sodium benzoate, 0.025% (0.00025 g/ml) strawberry aroma, 0.35% (0.0035 g/ml) citric acid, 64% (0.64 g/ml) hydrogenated glucose syrup, purified water, in sufficient quantity up to 1 ml, and optionally one or more Sweetening or moisturising agents.

21. The formulation in accordance with claim 20 contains as Sweetener 0.2% (0.002 g/ml) of sodium saccharin and as moisturizer 10% (0.1 g/ml) of glycerol.

22. Pharmaceutical formulations in accordance with claim 2 for use in the treatment of short-term and long-term memory disorders.

23. A method for the treatment of short- and long-term memory disorders where the method involves the step of administering to a subject with such disorders a therapeutically effective amount of the extract in accordance with claim 1 or any pharmaceutical formulation in accordance with claim 2.
